(19) United States
(12) Patent Application Publication
(10) Pub. No.: US 2026/0355138 A1
(43) Pub. Date: Nov. 12, 2026

(54) TOPICAL COMPOSITION AND USE THEREOF TO REDUCE POST-SURGICAL USE OF OPIOIDS

(71) Applicant: KAZM Pharmaceuticals Inc., Hamilton (CA)

(72) Inventors: Patrick Musitano, Hamilton (CA); Paul Zalzal, Hamilton (CA)

(21) Appl. No.: 19/705,405

(22) Filed: Jun. 11, 2026

Related U.S. Application Data
(63) Continuation of application No. PCT/CA2024/051639, filed on Dec. 10, 2024.
(60) Provisional application No. 63/608,628, filed on Dec. 11, 2023.

(51) Int. Cl. A61K 31/192; A61K 9/00; A61K 31/167; A61K 31/197; A61K 31/485; A61K 47/10; A61K 47/18; A61K 47/24; A61K 47/26; A61K 47/36; A61K 47/44; A61P 29/00

(52) U.S. Cl. CPC A61K 31/192; A61K 9/0014; A61K 31/167; A61K 31/197; A61K 31/485; A61K 47/10; A61K 47/183; A61K 47/24; A61K 47/26; A61K 47/36; A61K 47/44; A61P 29/00

(57) ABSTRACT

The topical composition is for application to a portion of skin at a locus of postsurgical pain and comprises: an amount of anti-inflammatory analgesic; an amount of muscle relaxant; an amount of neuropathic analgesic; an amount of anesthetic; and a carrier having skin penetration enhancement qualities.

### CROSS-REFERENCE TO RELATED APPLICATIONS

This application claims priority to U.S. Provisional Patent Application Ser. No. 63/608,628, filed Dec. 11, 2023, and incorporated herein by reference.

### FIELD OF THE INVENTION

The present invention in general relates to a topical composition and method of use, and in particular, to a topical composition containing anesthetic and analgesic and use thereof to treat post-surgical pain and reduce the post-surgical use of opioids.

### BACKGROUND OF THE INVENTION

The use of both natural and synthetic compounds, such as aspirin, ibuprofen, naproxen, a variety of herbal remedies, corticosteroids, and opioids for the treatment of pain are well known. Although widely used, these compounds cause many negative side effects such as gastric irritation, ulceration, nausea, lowered blood pressure, respiratory depression, itching, and skin rashes.

Chronic pain is generally defined as any pain which lasts more than twelve weeks and often lasts months or even years. In contrast, acute pain is the natural sensation that alerts of the presence of an injury or illness. With acute pain, the pain progressively becomes less severe as the injury heals or illness is fought-off. However, chronic pain exists when pain signals continue to be sent to the brain even after the initial cause of the acute pain is addressed. Chronic pain can also exist wholly without any initial cause of acute pain. Chronic pain one of the most pervasive health problems worldwide. In the United States, chronic pain can affect as many as eight of every ten adults. Common types of chronic pain include headaches, back pain, joint pain, and nerve pain. There is a growing appreciation that the side effect profiles of treatment of chronic pain with over-the-counter analgesics, opioids, and other conventional medications render these as unsuitable for long term pain management options.

Topical pain creams often consist of an anti-inflammatory, an analgesic, an antispasmodic, and a local anesthetic. One example of a topical pain cream is Multiprofen CC™. Topical delivery systems are used to control pain and act locally on damaged or dysfunctional soft tissues or peripheral nerves. They are not absorbed through the gastrointestinal system and do not undergo first pass hepatic metabolism. Less than 5% of the dose that is topically applied reaches the systemic circulation. (Osterwalder et al., 2002.) Pain signaling involves complex mechanisms. For neuropathic pain, peripheral activity of afferent nerves is an important contributor to pain. These conditions can benefit from a localized application of topical medications; combinations of agents targeting different pain mechanisms may be particularly useful. It is postulated that topical medications may play a role in desensitizing trigger zones and can reduce peripheral sensibility of areas affected by deafferentation and neuroma formation. (Sardana et al., 2017).

Opioids are very commonly prescribed for postsurgical pain, but they can cause a host of unwanted side effects. For example, total knee arthroplasty (TKA) is a surgical treatment which involves replacing the damaged articular cartilage of the knee joint with an artificial prosthetic in end-stage knee osteoarthritis. Typically, opioids such as Hydropmorphone and Hydromorphone Contin are prescribed as a standard post operative regime. Although TKAs are mostly successful, approximately 1 in 5 patients are unsatisfied with their outcomes with 16-33% of patients of patients experiencing lasting pain following TKA. Notably, orthopedic surgeons prescribe opioids more frequently than in any other surgical specialty. This is a rising concern to the healthcare system as it may promote the development of opioid use disorder.

Accordingly, there is a need for alternative therapies to opioid prescribing in patients having surgeries, such as knee, to reduce the health risks associated with opioid medications.

### DETAILED DESCRIPTION OF THE INVENTION

An inventive topical composition and method of treating postsurgical pain while reducing the amount of opioids used by a postsurgical patient are provided. The inventive topical composition contains ketoprofen, baclofen, amitriptyline, lidocaine and a carrier having skin penetration enhancement qualities. The inventive method for treating postsurgical pain and reducing the amount of opioids used by a postsurgical patient includes applying a topical composition that contains ketoprofen, baclofen, amitriptyline, lidocaine and a carrier having skin penetration enhancement qualities to a surgical incision in conjunction with standard care post-surgical regimes including provision of a standard care pain medication, such as an opioid.

According to embodiments, the topical composition used in the inventive method is the “topical composition for pain relief” disclosed in U.S. Pat. No. 9,707,197, which is hereby incorporated by reference. According to another embodiment, the topical composition is identical to the “topical composition for pain relief in U.S. Pat. No. 9,707,197 but for the Permeabase™ carrier, which is modified as detailed hereinafter. According to some embodiments, a subject treated according to an inventive method consumes significantly less of the provided standard care pain medication as compared to a postsurgical patient treated without the inventive topical composition. The present invention is well suited to treat postsurgical pain with significantly reduced opioid use in order to avoid the negative side effects caused by commonly used anti-inflammatory and pain-treating compounds.

It is appreciated that in addition to the treatment of postsurgical human pain, an inventive topical composition is readily applied to therapeutic effect to animal skin to treat pain. By way of examples such animals illustratively include dogs, cats, horses, cattle, sheep, primates, or rodents.

It is to be understood that in instances where a range of values are provided that the range is intended to encompass not only the end point values of the range but also intermediate values of the range as explicitly being included within the range and varying by the last significant figure of the range. By way of example, a recited range from 1 to 4 is intended to include 1-2, 1-3, 2-4, 3-4, and 1-4.

An inventive topical composition is provided in a lipophilic ointment base or formed into an oil in water emulsion that contains ketoprofen in an amount of 5 to 10 total weight percent of the composition, baclofen in an amount of 0.1 to 10 total weight percent of the composition, amitriptyline in an amount of 0.1 to 5 total weight percent of the composition; lidocaine in an amount of 0.1 to 10 total weight percent of the composition, an optional skin penetration enhancer in an amount of 0 to 10 total weight percent of the composition, and a carrier present in an amount making up a remainder of the composition.

Ketoprofen has anti-inflammatory activities. It is well documented through numerous trials that Ketoprofen is effective topically. Ketoprofen applied on the skin is able to enter tissues, reaching concentrations greater than in plasma, and producing the desired pharmacological activity, whereas plasma concentrations are too low to produce systemic activity or side effects. Studies in the literature have shown that by applying a 100 mg Ketoprofen patch daily directly to the focus of the painful condition, the concentration of Ketoprofen in the affected area was anywhere from 6 to 354 times higher than in the plasma. (Osterwalder et al., 2002). Topical applications of Ketoprofen allow the attainment of high intra-articular tissue concentrations, with limited systemic exposure (Rolf et al., 1999). Topical application delivered Ketoprofen to the target tissues at a similar concentration to that observed after oral administration without a high plasma concentration. Topical application rapidly achieved effective tissue levels and was maintained for at least 20 hours (Sekiya et al., 2010).

Baclofen is a GABA agonist myorelaxant possessing presynaptic depressant action at NMDA and non-NDMA receptors. Activation of GABAb receptors by local administration of Baclofen results in a uniform reduction in formalin-evoked behaviours (Zhou et al., 1998), and these receptors may represent a more promising target than GABAa receptors. (Sawynok, 2003).

Local release of adenosine and activation of adenosine A1 receptors is involved in the action of Amitriptyline, as analgesia is reduced by adenosine receptor antagonists and local administration of Amitriptyline enhances the peripheral availability of adenosine. (Sawynok et al., 1999), (Esser & Sawynok, 2000), (Liu et al., 2000).

Lidocaine is a local anesthetic which acts by blocking sodium channels, preventing nerve depolarization and stopping nerve signals (Heir et al., 2008). A randomized, double-blind, vehicle-controlled study demonstrated that topical Lidocaine reduces the intensity of all common neuropathic pain qualities and thus may be of potential benefit for nonallodynic neuropathic pain states (Galer et al., 2002).

In some inventive embodiments, a skin penetration enhancer is present from among propylene glycol, benzyl alcohol, and 2-(2-ethoxyethoxy) ethanol and are each individually typically present in amounts of from 0% to 10% of the total weight of the composition. Skin penetration enhancers are appreciated to be particular useful in transdermal patches impregnated with an inventive composition to provide sustained dosing to a locus of pain.

According to embodiments, the carrier comprises at least one of petroleum jelly, paraffin, lanolin, menthol, or beeswax. In another embodiment the carrier an inventive topical composition comprises at least one of petroleum jelly, paraffin, menthol, and combinations thereof. In certain embodiments of an inventive topical composition the carrier is in the form of a serum, a cream, a spray, or an ointment. In other inventive embodiments, the inventive topical composition may be in the form of a paste, gel, lotion, powder, aerosol, or liquid. In some inventive embodiments the carrier comprises an oil. Oils suitable for suspension or dissolution of the topical composition are limited only by storage stability with the ingredients and skin compatibility. Oils operative herein illustratively include plant based oils such as olive oil, grapeseed oil, almond oil, jojaba oil, safflower oil, corn oil, peanut oil, sesame oil, cannola oil, soy oil, soybean oil, burdock root oil, tea tree oil, coconut oil, apricot seed oil, walnut oil, and combination thereof; and animal based oils such as shark liver oil, cod liver oil and fish liver oil; and combinations thereof. In certain inventive embodiments, the carrier oil is selected to operate synergistically with the ingredients of the composition by imparting therapeutic properties. By way of non-limiting example apricot kernel oil has the attributes on human skin of being anti-inflammatory and hypoallergenic.

In some inventive embodiments, an essential oil is provided. The essential oil illustratively includes Peppermint (Mentha piperita), Spearmint (Mentha spicata), Eucalyptus (Eucalyptus globulus), Lavender (Lavender officinalus), Orange Blossom (Citrus sinensis), Rosemary (Rosmarinus officinalis), Aloe (Aloe Vera), Myrrh (Commiphora myrrha), Frankincense (Boswellia carteri), Clove (Syzygium aromaticum), Chamomile (Matricaria chamomilla), Majoram (Origanum majorana), Yarrow (Achillea millefolium), Sandalwood (Santalum album), Ginger (Zingiber officinale), Clary Sage (Salvia sclarea), Juniper (Juniperus communis), Cajuput (Melaleuca leucadendra), Camphor (Cinnamomum camphora), Cinnamon (Cinnamomum verum), and combinations thereof. An essential oil, or combination of essential oils, if present, are present from 0.1 to 19 total weight percent of the composition. Lavender (Lavender officinalus) and Orange Blossom (Citrus sinensis) appear to be particularly advantageous, each alone, or in combination.

Peppermint extract contains among other constituents menthol (40.7 wt. % of the extract) and menthone (23.4%), menthyl acetate, 1,8-cineole, limonene, beta-pinene carvone, jasmone, carvacrol, limonene, phellandrene, and beta-caryophyllene. Menthol has a pain cessation cooling effect on skin. Peppermint extract also has anti-inflammatory properties.

Spearmint extract contains among other constituents limonene, dihydrocarvone, 1,8-cineol, and menthol. Spearmint extract has anti-inflammatory properties, reduces swelling due to nerve and muscle pain, and reduces pain associated with arthritis.

Eucalyptus extract contains among other constituents 1, 8-cineole pinene, phellandrene, and limonene. 1, 8-cineole is present in an amount of at least 70 total weight percent and confers antibacterial, expectorant, decongestant, and anti-inflammatory properties.

Lavender extract contains linalool, perillyl alcohol, linalyl acetate, camphor, limonene, tannins, triterpenes, coumarins, cineole, and flavonoids. The skin quickly absorbs lavender oil. Lavender extract has anti-inflammatory, antifungal, antibacterial, antiseptic, analgesic, and anxiolytic properties. Lavender extract can also be used as a scenting agent in some embodiments of an inventive topical composition.

Orange blossom extract is made from neroli oil and can also be used as a scenting agent in some embodiments of an inventive topical composition.

Rosemary extract contains cuminic acid, bornyl acetate, caryophyllene, monoterpene hydrocarbons (alpha and beta-pinene), camphene, limonene, camphor, borneol, cineole, linalool, and verbinol. Rosemary contains a wide variety of volatile and aromatic components. Flavonoids in the extract include diosmetin, diosmin, genkwanin, luteolin, hispidulin, and apigenin. Phenols in rosemary extracts include caffeic, chlorogenic, labiatic, neochlorogenic, and rosmarinic acids, as well as salicylates. Without intending to be bound to a particular theory, rosemary extracts in the present invention confer antimicrobial, anti-cancer and anti-oxidative effects. Carnosol and carnosic acid are believed to account for more than 90% of the antioxidant properties of rosemary extract. Rosemary also exhibits anti-inflammatory and pain-relieving properties.

Aloe contains Arachidonic acid, linolenic acid, steroids (campestrol, cholesterol, (3-sitosterol), triglicerides, triterpenoid, glucomannan acemannan, aloeride, gibberillin, lignins, potassium sorbate, salicylic acid, bradykininase, maloyl glucan, veracylglucans, aloe-emodin, and uric acid. Without intending to be bound to a particular theory, aloe present in some embodiments of the present invention confers anti-inflammatory effects. Also, the polysaccharide glucomannan is an effective human skin moisturizer. Acemannan accelerates wound healing. Bradykininase, contributes anti-inflammatory properties and magnesium lactate, antipruritic effects. Salicylic acid and other antiprostaglandin compounds contribute local anti-inflammatory activity. Maloyl glucans also likely act as anti-inflammatories. Veracylglucans confer anti-proliferative effects. Aloe-emodin is thought to inhibit endothelial cell proliferation and inhibits tumor cell proliferation.

Myrrh extract contains myrrholic acid, cinnamic acid, cuminic acid, eugenol, cadinene, pinene, dipentene, heerabolene, limonene, furanodiene-6-one and methoxyfuranoguaia-9-ene-8-one. Without intending to be bound to a particular theory, myrrh extracts present in some embodiments of the present invention confer anti-tumoral effects and anti-microbial effects. Myrrh extract also has cytotoxic, analgesic, and anti-inflammatory properties.

Frankincense extract contains sesquiterpene, camphene, dipentene, pinene, phellandrene, olibanol, boswellic acid (3-alpha-hydroxy-urs-12-en-23-oic acid), volatile oils, terpinols, arabinose, xylose, galactose, uronic acids, beta-sitosterin and phlobaphenes. Without intending to be bound to a particular theory, frankincense extracts in the present invention confer anti-inflammatory effects and include specific inhibitors of 5-lipoxygenase, the key enzyme of leukotriene biosynthesis.

Clove extract contains eugenol, acetyl eugenol, beta-caryophyllene, methyl salicylate, pinene, and vanillin. Clove extract has anti-inflammatory and pain-relieving properties, particularly imparted by the beta-caryophyllene and methyl salicylate.

Chamomile extract contains α-pinene, β-pinene, sabinene, myrcene, caryophyllene, 1,8-cineol, terpinene, propyl angelate, and butyl angelate. Without intending to be bound to a particular theory, chamomile extracts present in some embodiments of the present invention confer anti-inflammatory and pain-relieving effects.

Majoram extract contains sabinene, alpha-terpinene, gamma-terpinene, cymene, terpinolene, linalool, sabinene hydrate, linalyl acetate, terpineol, and gamma terpineol. Majoram extract has analgesic, antispasmodic, antiseptic, antibacterial, antiviral properties. Majoram also functions as an anxiolytic, expectorant, nervine, sedative, and vasodilator.

Yarrow extract contains tricyclene, alpha-pinene, camphene, beta-pinene, sabinene, borneol acetate, 1,8-cineole, terpinene, limonene, isoartemisia ketone, and borneol. Yarrow extract promotes healing of the skin, reduces swelling in inflamed wounds, and treats gastrointestinal problems. Yarrow extract also has anxiolytic and anti-inflammatory properties.

Sandalwood extract contains sesquiterpenic alcohols including tricyclic alpha-santalol and beta-santalol. Sandalwood extract is an anti-inflammatory.

Ginger extract contains among other constituents camphene, beta-phellandrene, alpha-pinene, geranial, zingiberene, beta-bisabolene, beta-sesquiphellandrene, and curcumene. Ginger extract has anti-inflammatory properties particularly imparted by the camphene, pinene, beta-bisabolene, and curcumene present. Ginger extract is also a bronchodilator, an antioxidant, an antiviral, an antiseptic, and an analgesic.

Clary sage extract contains among other constituents linalyl acetate, linalool, germacrene, alpha-terpineol, and sclareol. Clary sage extract is an anti-inflammatory with a cooling property that soothes skin presenting inflammation. Clary sage extract also promotes wound healing, treats muscle aches, joint pain, and enhances circulation.

Juniper extract contains alpha-pinene, camphene, beta-pinene, sabinene, myrcene, alpha-phellandrene, terpinene, 1,4-cineole, beta-phellandrene, cymene, terpinen-4-ol, bornyl acetate, cayophyllene and trace amounts of limonene, camphor, linalool, linalyl acetate, borneol, and nerol. Juniper extract is an anxiolytic, an anti-inflammatory, treats skin irritation, and promotes fluid retention.

Cajuput extract contains among other constituents viridiflorol and 1,8-cineole. Cajuput extract has anti-inflammatory and pain-relieving properties. It is particularly effective at treating muscle pain.

Camphor extract contains among other constituents alpha-pinene, camphene, limonene, 1,8-cineole and cymene. Camphor extract is an anti-inflammatory and imparts a cooling effect to the skin. It is particularly effective in treating skin irritation such as sores, insect bites, itching, and rashes. It also helps to relieve pain caused by sore muscles. Camphor extract also has antibacterial, antiviral, and antifungal properties. Camphor extract also promotes circulation, has anxiolytic properties, and can reduce the intensity of convulsions, spasms, and muscle contractions.

Cinnamon extract contains among other constituents cinnamaldehyde, cinnamyl acetate, eugenol, and eugenol acetate. The cinnamaldehyde imparts antifungal, antibacterial, and antimicrobial properties. The cinnamyl acetate enhances circulation. The eugenol imparts antiseptic, anti-inflammatory, and analgesic properties. It also can reduce gastric and ulcer related pain. The eugenol acetate imparts antioxidant properties.

In some inventive embodiments, vitamin E, if present, is present from 0.1 to 3 total weight percent. Vitamin E or a derivative thereof has anti-inflammatory properties, promotes wound healing, and treats skin irritation. Vitamin E also boosts immune function, promotes eye health, and may lower the risk of cancer. It is appreciated that Vitamin E esters and salts are operative herein, as detailed in Z. A. Al-Talla and L. T. Tolley, “Analysis of vitamin E derivatives in serum using coordinated ion spray mass spectrometry” Rapid Commun Mass Spectrom. 2005; 19 (16): 2337-42.

In one embodiment an inventive topical composition comprises at least one of an adjuvant, a steroidal anti-inflammatory such as prototypical cortisone, an anti-bacterial such as silver sols, or a cellular regrowth stimulating substance such as biotin or epidermal growth factor. Illustrative examples of acceptable adjuvants include waxes; paraffins; starch; tragacanth; a pluronic; cellulose and cellulose derivatives; silicones; bentonites; silicic acid; talc; zinc oxide; aluminum hydroxide; calcium silicates; alginate; acrylate; hyaluronic acid; polyethylene glycol; vitamins such as ascorbic acid glucosides; partial glycerides of fatty acids; chitosan; and mixtures thereof.

In addition, some embodiments of the inventive topical composition may contain one or more compounds for improving cosmetic acceptability, including but not limited to, preservatives, humectants, fragrances, coloring agents, emollients, fillers, and the like.

A preservative is included in some inventive compositions at a concentration effective to inhibit undesirable effects such as microbial growth, UV and/or oxygen-induced breakdown of composition components, and the like. A preservative operative in an inventive gel is any of those known in the art and compatible with the components of an inventive composition. Examples include butylated hydroxytoluene (BHT) used as an antioxidant and edetate disodium (EDTA) used as an antioxidant synergist. When a preservative is included, it is present at concentrations sufficient to confer a preservative effect. In the case of BHT the typical range is from 0.01 to 0.03% and in the case of EDTA the typical range is from 0.005 to 0.1%.

A humectant has emollient properties. Humectants operative herein illustratively include glycerin, capric/caprylic triglyceride, vegetable oils, aloe barbadensis, and combinations thereof. A humectant is included at concentrations ranging from 0 to 10 total weight percent.

The preparation of the composition according to the invention includes bringing the components of the topical composition together in determined and precise proportions and in determined and precise forms, and this causes a synergy in therapeutic effect as to treating postsurgical pain with reduced opioid use. The inventive topical composition obtained gives effects and results which are considerably improved in comparison with the effects and results obtained with each constituent taken in isolation and successively or in a cumulative manner, and this is unexpected and surprising based on the prior art. The inventive composition also gives these improved effects and results without the negative side effects commonly experienced with prior art postsurgical pain treatments.

Because the inventive topical composition is applied to compromised skin in some inventive embodiments, all or some of the constituents of certain embodiments of an inventive composition are sterilized, either individually or together. Any suitable sterilization method or combination of sterilization methods can be used and can be used on any combination of composition and/or packaging components, as long as the sterilization does not adversely affect the therapeutic effectiveness of the inventive topical composition. By way of non-limiting example, suitable sterilization techniques that may be employed include dry and moist heat sterilization, ionizing radiation (such as electron beam or gamma irradiation), exposure to gas, and aseptic filtration. It is appreciated that heat sterilization has a propensity to damage many plant extract components such as carbohydrates and proteins and as a result should be used with caution to avoid the loss of therapeutic effect. Where one or more of the above-described additives are present in the inventive composition, and where the other components of the composition are sterilized as described above, it is preferred that the additional additives also be sterilized as described above, so that sterility of the entire composition can be maintained.

A method of treating postsurgical pain in a subject includes providing an available amount of standard care pain medication to the subject for management of postsurgical pain, the available amount of standard care pain medication having a known total Morphine Equivalent Dose (MED) and applying a dose of a topical composition to a portion of skin of the subject at a locus of the postsurgical pain at least once in a 24 hour period for a treatment period, wherein the topical composition comprises ketoprofen, baclofen, amitriptyline, and lidocaine, as described above.

According to embodiments, the standard care pain medication is an opioid and is provided in according to a standard care post operative regime according to what is standard for a given surgery. For example, the post operative care regime is to provide Hydromorphone 1-2 mg po q4 h prn, dispensed as 60, 1 mg dose tabs and Hydromorphone Contin 3 mg po BID, dispensed as 14, 3 mg dose tabs, with refills available upon request.

According to embodiments, use of a topical composition comprising ketoprofen, baclofen, amitriptyline, and lidocaine, as described herein in conjunction with a standard care post operative regime for a given surgery reduces the amount of opioids consumed by the subject patient. That is, according to embodiments, a first MED amount of the standard care pain medication that is consumed by the subject during the treatment period is less than a second MED amount of the standard care pain medication consumed by a second subject not treated with the topical composition over a timer period equivalent to the treatment period. According to embodiments, the MED amount of the standard care pain medication that is consumed by the subject during the treatment period is at least 15 percent less than a MED amount of the standard care pain medication consumed by a second subject not treated with the topical composition over a timer period equivalent to the treatment period.

According to embodiments, the dose of topical composition is 1 to 5 grams of the topical composition. According to embodiment, a dose of the topical composition is applied to the locus of the postsurgical pain three times in a 24 hour period for a treatment period. According to embodiments, the treatment period is three months. According to embodiments, the topical composition used with the inventive method is a composition that comprises ketoprofen present in an amount of 10 total weight percent, baclofen present in an amount of 5 total weight percent, amitriptyline present in an amount of 2 total weight percent, and lidocaine present in an amount of 5 total weight percent, such as Multiprofen CC™.

An inventive topical composition may be applied to the skin at a locus of the postsurgical pain. In certain inventive embodiments postsurgical pain will begin to subside within fifteen minutes of applying of an inventive topical composition to the skin at a locus of the postsurgical pain. In some inventive embodiments a single application of an inventive topical composition maintains therapeutic effect for up to three hours. For further protection and to ensure the inventive composition is fully absorbed into the skin, it is appreciated that a covering is readily applied over the skin covered by the inventive composition. Any suitable covering may be used, and may be tailored to the location, type, and severity of the postsurgical pain. By way of illustrative example, suitable coverings include bandages, wound dressings, gloves, and the like. In certain inventive embodiments a transdermal patch is impregnated with an inventive composition. The transdermal patch impregnated with an inventive composition is affixed to the skin at a locus of the postsurgical pain.

According to embodiments, the topical composition is applied by a person or by the subject who is wearing gloves or using an applicator tool in order to avoid numbing effects in areas other than the locus of the postsurgical pain on the subject.

The present invention is further detailed with respect to the following non-limiting example.

### Example

A topical composition was procured, having the following active ingredients, by weight, in a Permeabase™ carrier: 5% Lidocaine; 10% Ketoprofen; 5% Baclofen and 2% amitriptyline.

The ingredients of Permeabase™ carrier are shown in Table 1

TABLE 1

| n | CAS | Name | Common Name | Proper name | Range | Conc % | Qty |
|---|---|---|---|---|---|---|---|
| 1 | 7732-18-5 | Water | | Water | 1 | 50-100 | 67.6 |
| 2 | 8043-29-6 | Glycerin | | 1,2,3-Propanetriol | 3 | 3-10 | 5 |
| 3 | 11138-66-2 | Xanthan Gum | | Xanthan Gum | 6 | 0.1-0.3 | 0.1 |
| 4 | 6381-92-6 | Disodium EDTA | | Acetic acid, (ethylenedinitrilo)tetra-, disodium salt, dihydrate | 6 | 0.1-0.3 | 0.1 |
| 5 | 73398-61-5 | Fractionated coconut oil | | Caprylic/Capric Triglyceride | 3 | 3-10 | 5 |
| 6 | 142-91-6 | Isopropyl myristate | | Isopropyl myristate | 3 | 3-10 | 7 |
| 7 | 67762-27-0 | Promulgen-D | Cetearyl Alcohol | Cetostearyl alcohol | 3 | 3-10 | 2 |
| | 68439-49-6 | | Ceteareth-20 | PEG-20 Cetyl/Stearyl Ether | | | |
| 8 | 67762-27-0 | Polawax | Cetearyl Alcohol | Cetostearyl alcohol | 3 | 3-10 | 10 |
| | 9004-99-3 | | PEG-150 Stearate | Polyoxyethylene (150) Monostearate | | | |
| | 9005-67-8 | | Polysorbate 60 | Polyoxyethylene 20 sorbitan monostearate | | | |
| | 9005-00-9 | | Steareth-20 | Polyoxyethylene (20) Stearyl Ether | | | |
| 9 | 36653-82-4 | Cetyl Alcohol | | 1-Hexadecanol | 4 | 1-3 | 2 |
| 10 | 8029-76-8 | Hydroxylated Lecithin | | 1,2-Diacylglycero-3-phosphocholine | 6 | 0.1-0.3 | 1 |
| 11 | 122-99-6 | Phenoxyethanol | | 1-Hydroxy-2-phenoxyethane | 5 | 0.3-1.0 | 0.1 |
| 12 | 57-55-6 | Liquid | Propylene Glycol | 1,2-Dihydroxypropane | 5 | 0.3-1.0 | 0.1 |
| | 78491-02-8 | Germall | Diazolidinyl Urea | N-[1,3-Bis(hydroxymethyl)-2,5-dioxo-4-imidazolidinyl]-N,N′-bi | | | |
| | 55406-53-6 | Plus | Iodopropynyl Buty | 3-Iodo-2-propynyl butylcarbamate | | | |

| | |
|---|---|
| TOTAL: | 100 |

The carrier is prepared in accordance with Table 2

TABLE 2

| | |
|---|---|
| A | Slowly blend together #1, 2, 3 & 4 in a stainless steel |
| | jacket container. Heat to 70 °C using a lightnin mixer. |
| B | Mix together #5, 6, 7, 8, 9 & 10 in a separate stainless |
| | steel vessel. Heat to 70 °C. |
| C | Slowly combine phase #B to phase #A while continuing to |
| | mix using a lightin mixer. Begin cooling process. |
| D | When the temperature of phase #C reaches 50 °C, add #11 |
| | & then follow with #12. Continue to mix, cool to 40 °C. |
| E | Continue to cool blended batch to 35 °C. |

### Case 1

A 77 year old man with end stage knee arthritis underwent a left total knee replacement. His right knee had been replaced the previous year. He was prescribed the topical composition and given instructions to apply 1 gram of the topical composition to his knee three times daily in the immediate post operative period and for the first 2 weeks after surgery. On two week follow up, he reported his pain was well controlled. He reported that recovery from his left total knee replacement with the use of the composition was much easier than the recovery from his right total knee replacement he had done the previous year [in the absence of the topical composition].

### Case 2

A 76 year old woman underwent a right total knee replacement for the management of severely painful end stage osteoarthritis. She was discharged home the same day, with instructions to return to the hospital if she was unable to manage her pain at home. She was prescribed the topical composition and given instructions to apply 1 gram of the topical composition to her knee three times daily in the immediate post operative period and for the first 2 weeks after surgery. She was also prescribed standard oral analgesics. She did not return to the hospital for pain management at any time in her post operative period. At two week follow up, she reported that the use of the topical composition helped her manage her pain at home.

### Case 3

A 62 year old woman underwent a left total knee replacement surgery and was discharged home the same day. She had previously undergone a right total knee replacement surgery which was complicated by ongoing pain and instability, and ultimately required another surgical intervention to remedy the situation. She was prescribed the topical composition and given instructions to apply 1 gram of the topical composition to her knee three times daily in the immediate post operative period and for the first 2 weeks after surgery. She reported that the topical composition assisted with her pain control in the post operative period. She did not need to return to the hospital for pain control and upon follow up reported that she felt her left total knee replacement surgery was more successful than her previous right total knee replacement surgery [which was conducted in the absence of the topical composition].

### Case 4

A 66 year old man underwent a left total knee replacement surgery and was discharged home the same day. He was prescribed the topical composition and given instructions to apply 1 gram of the topical composition to his knee three times daily in the immediate post operative period and for the first 2 weeks after surgery. He was also prescribed oral analgesics. He was instructed to return to the hospital if he could not manage his post operative pain at home. He did not need to return to the hospital for pain control. At the two week follow up he reported that he did not require all the oral analgesics that he was prescribed and he felt the topical composition was helping reduce his post operative pain.

### Case 5

A 78 year old man underwent a right total knee replacement and was discharged home the same day. He was instructed to return to the hospital if he could not manage his pain at home. He was prescribed the topical composition and given instructions to apply 1 gram of the topical composition to his knee three times daily in the immediate post operative period and for the first 2 weeks after surgery. He was also prescribed the usual oral analgesic medications. At follow up he reported his pain had been well controlled in the post operative period and he did not need to return to the hospital after discharge for help with pain management. He felt that the topical composition was significantly helping his pain control in the post operative period.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient road map for implementing the exemplary embodiment or exemplary embodiments. It should be understood that various changes can be made in the function and arrangement of elements without departing from the scope as set forth in the appended claims and the legal equivalents thereof.

1. A topical composition for use in the treatment of postsurgical pain and for application to a portion of skin at a locus of postsurgical pain, the topical composition comprising:
an amount of anti-inflammatory analgesic;
an amount of muscle relaxant;
an amount of neuropathic analgesic;
an amount of anesthetic; and
a carrier having skin penetration enhancement qualities.

2. The composition according to claim 1, wherein the neuropathic analgesic is Amitriptyline, the muscle relaxant is Baclofen, the anesthetic is Lidocaine and the anti-inflammatory analgesic is Ketoprofen.

3. The topical composition of claim 2, wherein the amount of ketoprofen is 5 to 10 total weight percent.

4. The topical composition of claim 2, wherein the amount of baclofen is 0.1 to 10 total weight percent.

5. The topical composition of claim 2, wherein the amount of amitriptyline is 0.1 to 5 total weight percent.

6. The topical composition of claim 2, wherein the amount of lidocaine is 0.1 to 10 total weight percent.

7. The topical composition of claim 1, wherein the carrier comprises a diluent, a humectant, a thickener, an emulsifier and a preservative.

8. The topical composition according to claim 1, wherein the carrier comprises one or more of water, glycerin, Xanthan Gum, Disodium EDTA, fractionated coconut oil, isopropyl myristate, Promulgen-D, Polawax, Cetyl Alcohol, Hydroxylated Lecithin, Phenoxyethanol and Liquid Germall Plus.

9. The topical composition of claim 1, wherein the composition is provided as a cream.

10. A method of treating postsurgical pain in a subject, the method comprising:
providing an available amount of standard care pain medication to the subject for management of postsurgical pain, the available amount of standard care pain medication having a known total Morphine Equivalent Dose (MED); and
applying a dose of a topical composition to a portion of skin of the subject at a locus of the postsurgical pain at least once in a 24 hour period for a treatment period, wherein the topical composition comprises at least two of: ketoprofen, baclofen, amitriptyline, and lidocaine.

11. The method of claim 10 wherein a first MED amount of the standard care pain medication that is consumed by the subject during the treatment period is less than a second MED amount of the standard care pain medication consumed by a second subject not treated with the topical composition over a time period equivalent to the treatment period.

12. The method of claim 11 wherein the first MED amount is at least 15 percent less than the second MED amount.

13. The method of claim 10, wherein the standard care pain medication is an opioid.

14. The method of claim 10, wherein the standard care pain medication includes Hydromorphone 1-2 mg po q4 h prn, dispensed as 60, 1 mg dose tabs and Hydromorphone Contin 3 mg po BID, dispensed as 14, 3 mg dose tabs.

15. The method of claim 10, wherein the dose of topical composition is 1 to 5 grams of the topical composition.

16. The method of claim 10, further comprising applying at least two additional doses of the topical composition within the 24 hour period.

17. The method of claim 10, wherein the treatment period is three months post surgery.

18. The method of claim 10, wherein the topical composition comprises ketoprofen present in an amount of about 10 total weight percent, baclofen present in an amount of about 5 total weight percent, amitriptyline present in an amount of about 2 total weight percent, and lidocaine present in an amount of about 5 total weight percent.

19. The method of claim 10, wherein the topical composition is applied by a person or by the subject who is wearing gloves.
